(19) United States
(12) Patent Application Publication
(10) Pub. No.: US 2004/0254429 A1
(43) Pub. Date: Dec. 16, 2004

(54) Data storage device for integrating data of several medical measuring instruments

(75) Inventor: Paul Yang, Jhonghe City (TW)

Correspondence Address: TROXELL LAW OFFICE PLLC, SUITE 1404, 5205 LEESBURG PIKE, FALLS CHURCH, VA, 22041, US

(73) Assignee: Health & Life Co., Ltd.

(21) Appl. No.: 10/458,220

(22) Filed: Jun. 11, 2003

(51) Int. Cl. A61B 5/00

(52) U.S. Cl. 600/300

(57) ABSTRACT

The present invention discloses a data storage device for integrating the data of several medical measuring instruments, comprising a control unit which is coupled to a transmitting unit, a computation unit, a data storage unit, a display unit, and a reminding unit, such that when it is in use, the transmission unit will send the data measured by a medical measuring device to the control unit, and then save the data to a specified location of the data storage unit. Through the selection made externally, the specified data can be read from the data storage device, and sent and displayed on a display unit with a specified way. In the meantime, the computation unit will compare the data with the basic set value; if the data exceeds the basic set value, then the control unit will instruct the reminding unit to issue a warning to remind users.

### BACKGROUND OF THE INVENTION

1. Field of the Invention

The present invention relates to a data storage device for integrating the data of several medical measuring instruments, which determines and processes the data measured by the medical measuring instruments for measuring various health conditions (such as a sphygmomanometer, a glucose/cholesterol scale, a body water and fat scale, etc.), and then stores the data into a specified location. Such data storage device for integrating the data of several medical measuring instruments also can set up the time for reminding users to take medicines and the electronic business cards including the telephone number and address of medical staffs, which provides a convenient way for the users to carry the business card and to contact medical staffs. In the meantime, such arrangement can also provide a convenient way to let both users and medical staffs know about the user's health conditions and remind the users about the points for attention.

2. Description of the Related Art

In the present market, various machines for measuring health conditions such as a sphygmomanometer, a body fat scale, a glucose scale, and a cholesterol scale, etc. generally processes the data immediately after the measurement is taken, and displays the measuring results in a digital or analog way, but these machines usually cannot save the measuring result obtained by users as a reference for medical treatments or provide a reminder to users to take medicines in daily life. Even if some instruments can save the measuring results, the storage capacity of the data storage unit is limited and does not allow us to save the data for a long-term use. Furthermore, if various instruments are needed for the measurements (such as the sphygmomanometer, body fat scale, glucose scale, and cholesterol scale, etc), then the user has to take all these instruments to the hospital for the reference for medical staffs, which will be very inconvenient to users as well as to the medical staffs. Even worse, a patient may miss the important timing for having the medical treatment as needed.

Furthermore, due to the busy works in our daily life, patients often forget the time to take medicines. As a result, it will adversely affect the patient's conditions, or the patient has to look for his/her notebook or directory for the contact information of medical staffs when the patient wants to contact with the medical staffs. Thus, it is very inconvenient to users for the use of the prior-art devices.

In view of the shortcomings of the traditional medical measuring device according to the prior arts, the present inventor aimed at the problem and started finding a way for its improvement and overcoming its shortcoming. The present inventor based on years of experience accumulated from the engagement in the related industry conducted extensive research to resolve the aforementioned shortcomings and invented the present invention.

### SUMMARY OF THE INVENTION

The primary objective of the present invention is to provide a storage device for integrating the data of several medical measuring instruments, which comprises a control unit, and the control unit is connected to a transmission unit, a computation unit, a data storage unit, a display unit, a reminding unit, and a timing unit, so that the transmission unit will send the data measured by an external measuring device to the control unit, and save the data into a specification location of the data storage unit, and through a selection specified externally, the specified data will be read from the data storage unit and sent to and display on the display unit in a specified way. In the meantime, the computation unit will compare the data with the basic set value; if the data exceeds the basic set value, then the control unit will instruct the reminding unit to issue a warning to remind the user. Such arrangement can completely save various medical data of personal health measured by external measuring devices through the transmission, and also allows the users and medical staffs to know about the user's health condition anytime.

Another objective of the present invention is to provide a storage device for integrating the data of several medical measuring instruments, wherein the data storage unit has one or more data areas and a data buffer, so that when the data storage device is in use, the data from various measuring devices are saved in the data buffer first, and then individually saved into the corresponding data area after the computation unit finishes its processing.

A further objective of the present invention is to provide a storage device for integrating the data of several medical measuring instruments, wherein users can set up the time to reminding users to take medicines by selecting the time externally. When the set time is up, the control unit will issue a warning to remind the user to take medicines.

### BRIEF DESCRIPTION OF THE DRAWINGS

Other features and advantages of the present invention will become apparent in the following detailed description of the preferred embodiments with reference to the accompanying drawings, in which:

FIG. 1 is a block diagram of the data storage device of the present invention.

FIG. 2 is an illustrative diagram of the data storage device of the present invention.

FIG. 3 is a flowchart of saving data according to the present invention.

FIG. 4 is an illustrative diagram of setting up the time according to the present invention.

FIG. 5 is a flow chart of the setting up the time according to the present invention.

FIG. 6 is an illustrative diagram of displaying data according to the present invention.

FIG. 7 is a flow chart of displaying data according to the present invention.

FIG. 8 is a flow chart of reminding the patient to take medicines according to the present invention.

### DETAILED DESCRIPTION OF THE PREFERRED EMBODIMENTS

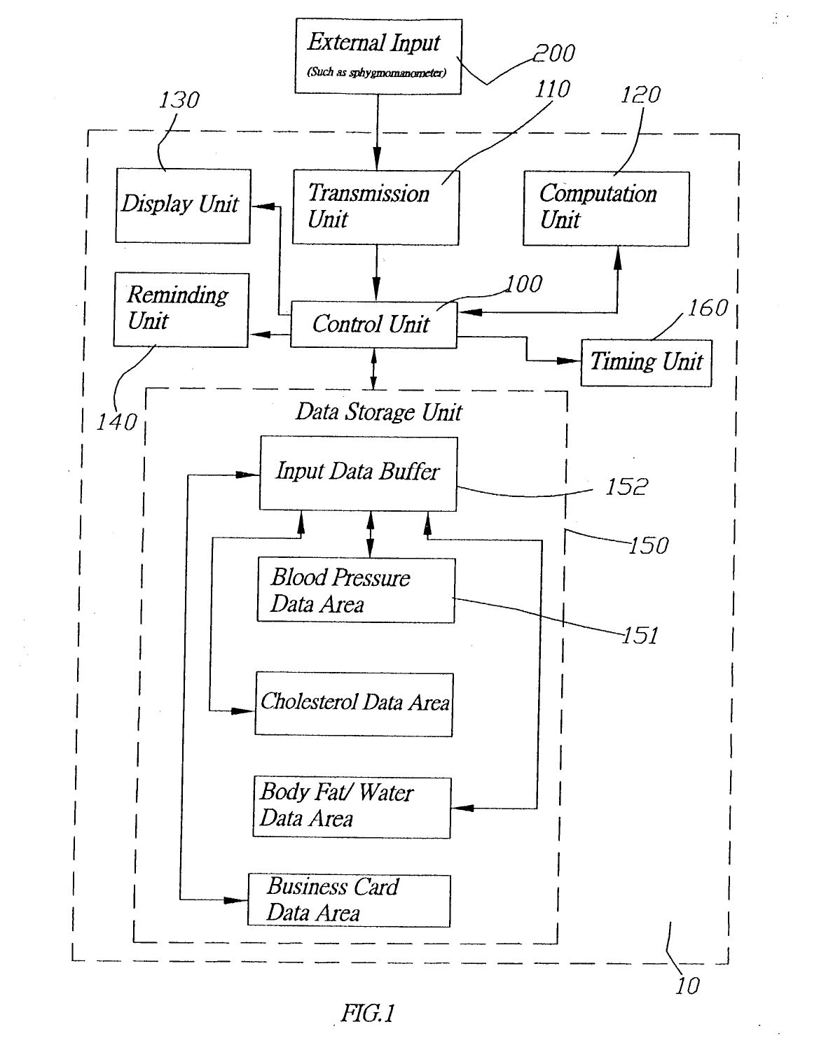

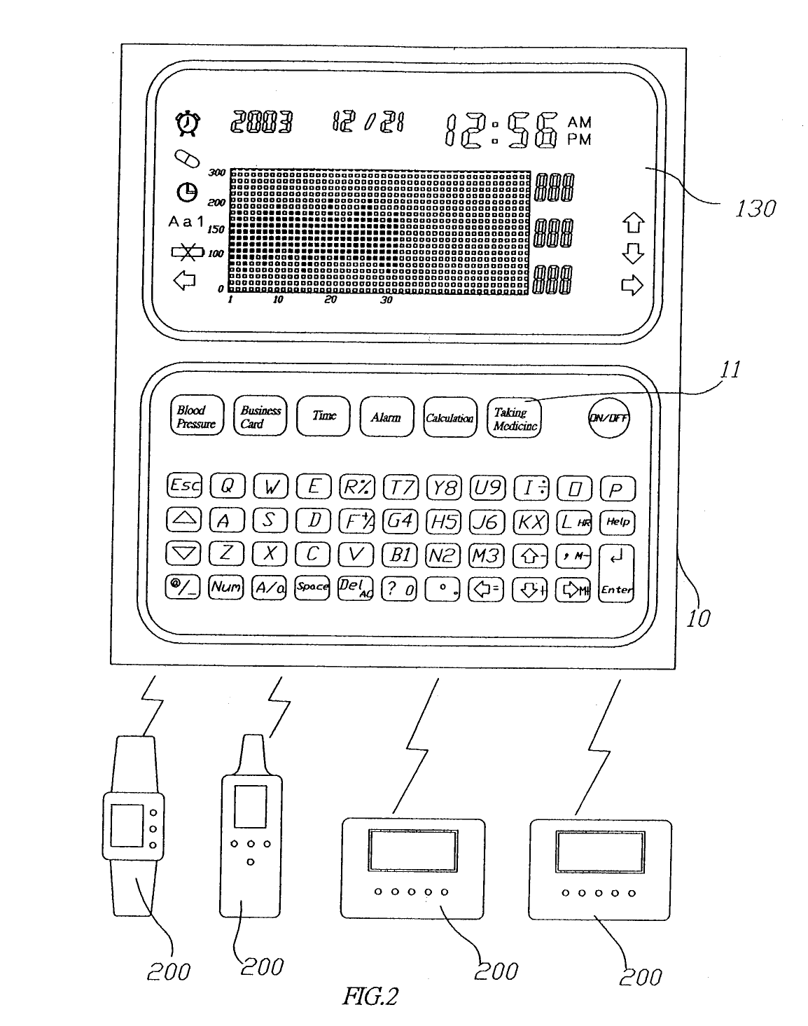

Please refer to FIG. 1 for the data storage device for integrating data of several measuring instruments, comprising a housing 10 (as shown in FIG. 2) with a control unit 100, which is a processor in this embodiment, and the control unit 100 is electrically coupled to a transmission unit 110, a computation unit 120, a display unit 130, a reminding unit 140, a data storage unit 150, and a timing unit 160 by a circuit.

The control unit 100 is used to control the action of each unit, and the transmission unit 110 is used to receive the data obtained from an external measuring device 200, and to send the data to the control unit 100. In this embodiment, the transmission unit 110 is an infrared (IR) transmission interface; however, the persons skilled in the art can use Ethernet, Universal Serial Bus (USB), serial bus interface, or wireless frequency interface. The external data is the data sent from a measuring device 200 for measuring various health conditions, and the measuring device 200 could be a sphygmomanometer, a body fat scale, a glucose scale, and a cholesterol scale, etc. These measuring devices 200 have an output unit to work together with the transmission unit 110, which is an infrared (IR) transmission interface as shown in FIG. 2. Further, the computation unit 120 is used to process/compare the data, which is a logical/computational unit in this embodiment. The display unit 130 is used to display data, which is a LCD screen in this embodiment as shown in FIG. 2. The reminding unit 140 is used to issue a warning to remind the user that the data of the measuring data entered exceeds the basic set value. The reminding unit 140 in this embodiment is an alarm buzz. In addition, the data storage unit 150 is used to store the data received from the transmission unit 110 or the data processed by the computation unit 120. The data storage unit 150 has one or more data areas 151 and one data buffer 152, and in this embodiment, the data storage unit is a memory. The timing unit 160 is used to calculate the time for the storage, and in this embodiment, the timing unit is a timer.

Further, the housing 10 comprises one or more press buttons 11, and these press buttons are electrically coupled to a power button, an input start button, various selection buttons, alphabetical button, and numeric button, etc. as shown in FIG. 2.

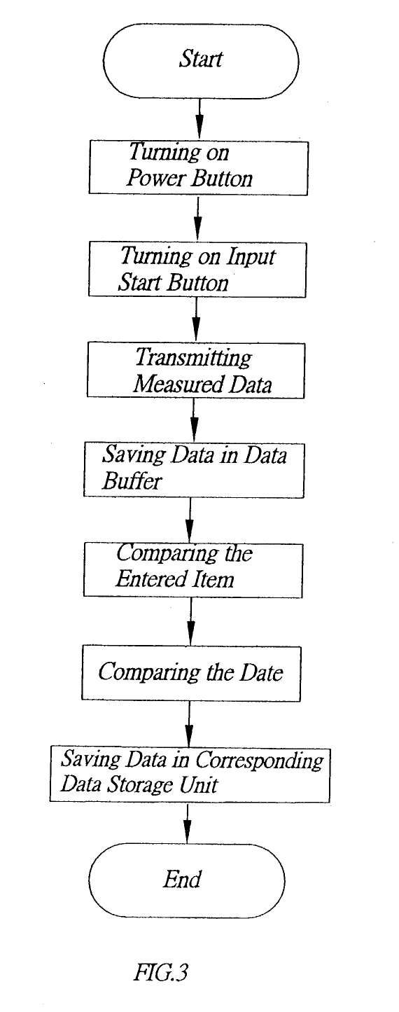

Please refer to FIGS. 2 and 3. When the measured data sent from the external measuring device 200 is received, the power button 11 on the housing 10 should be turned on, and then turn on the input start button 11 so that the output unit 110 of the external measuring device 200 is corresponsive to the transmission unit 110. After the connection, the transmission unit 10 will send the measuring data stored in the measuring device 200 to the control unit 100, and then to the data buffer 152 of the data storage unit 150. The index of the data will serve as a basis for determining the type of the measuring instrument; for example, 01 stands for the sphygmomanometer and 02 for the body fat scale, etc. After the control unit 100 checks the value to determine the type of measuring instrument, the control unit 100 will continue to compare the data with the one stored in the specified data area 150. If the data is determined as the most updated data, then such data will be saved in the corresponding specific data area 151 of the data storage device 150.

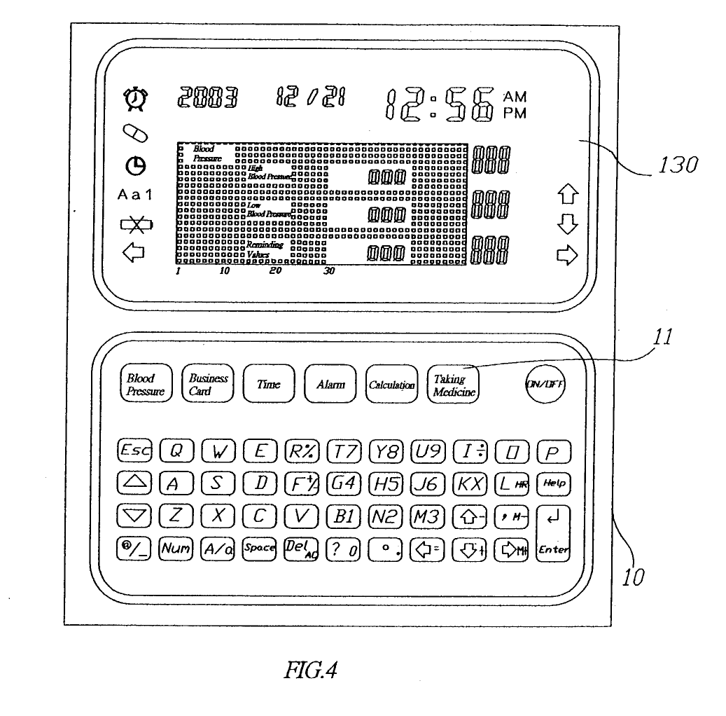

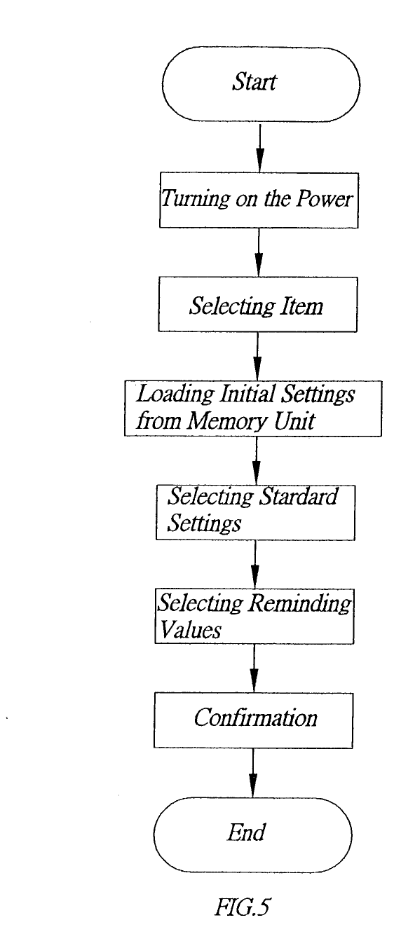

Further, please refer to FIGS. 4 and 5. When we set up the basic set values for various measuring data, the time for reminding user to take medicines, and the business cards, turn on the power button 11 first, and then select your desired item (such as the blood pressure data, the cholesterol data, the time for reminding patients to take medicines, and the business cards) by the setup button 11. While the control unit 100 is receiving the instruction of the selected item, the data storage unit 150 reads the initialized value and displays the data on the display unit 130. The user uses the alphabetical and numeric buttons to set up the desired basic set values, the reminding value, the time of reminding patients to take medicines, and enter the telephone number and address of the medical staff on the electronic business card. The setup will be completed after the data are confirmed.

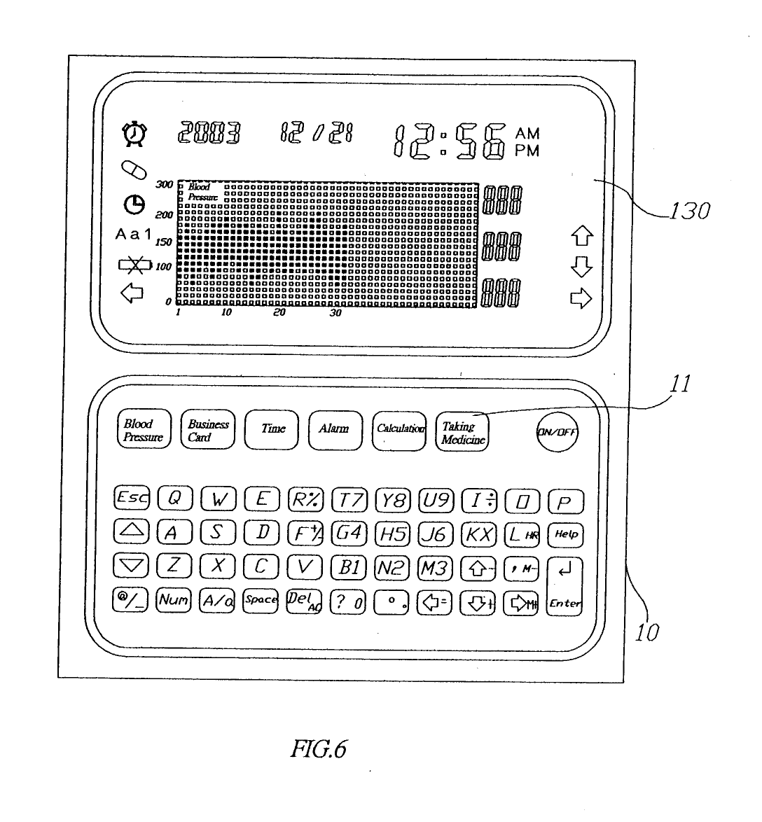

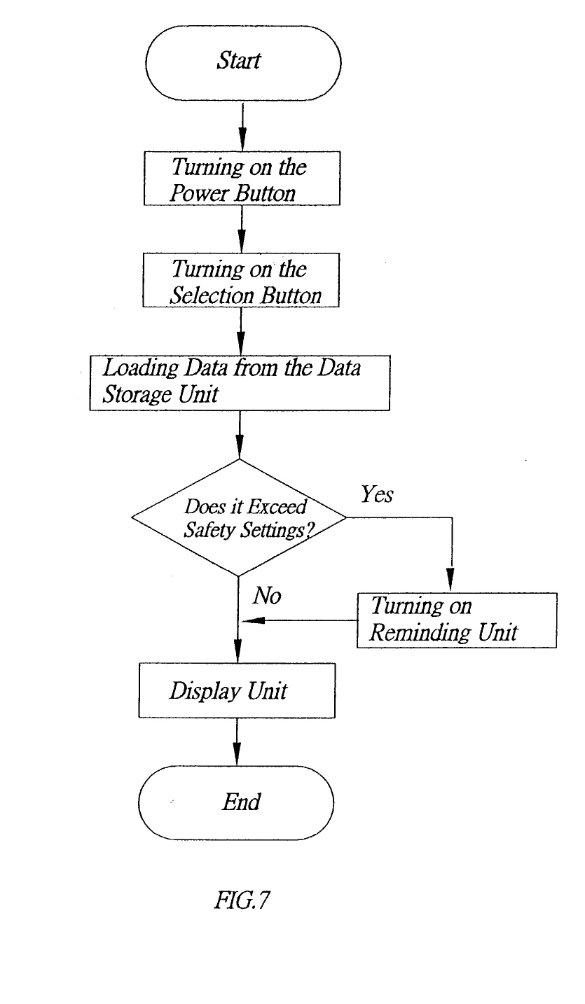

Further, please refer to FIGS. 6 and 7. When the data storage device is in use, turn on the power button 11 first, and then start the selection buttons to select your desired measuring data for the display. After the control unit 100 has received such instruction, it will read the related data from the specified data area 151 of the specified data. In the meantime, the computation unit 120 will compare the desired data for display with the basic set value; if the data exceeds the basic set value, then the reminding unit 140 is enabled to issue a warning reminder while displaying and blinking the data on the display unit. Such arrangement can bring up the user's attention regarding to the health conditions of the user.

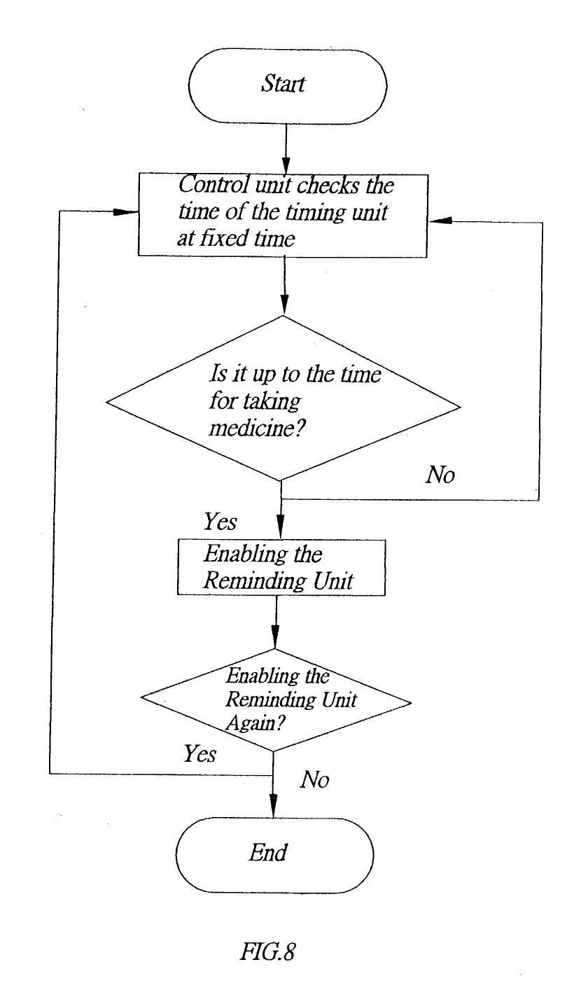

Further, please refer to FIGS. 1 and 8. When the user has preset the reminding time for taking medicine, the control unit 100 will start the sorting program and check the timing unit 160 if it is the time to take medicine; if it is not the time yet, then the control unit 100 will continue to check the time; if it is the time, then the control unit 100 will drive the reminding unit 140 to send out a message to remind the user to take medicines.

Additionally, please refer to FIG. 1 for the flow chart saving data according to the present invention. Such arrangement not only can completely save various data of personal health conditions, but also can allow individuals and medical staffs to know about the user's health conditions anytime.

In summation of the above description, the data storage device for integrating the data of several medical measuring instruments definitely overcomes the shortcomings of the prior art and enhances the performance and utility of the conventional data storage device and is submitted to the Patent and Trademark Office for review and granting of the commensurate patent rights.

While the present invention has been described in connection with what is considered the most practical and preferred embodiment, it is understood that the invention is not limited to the disclosed embodiments but is intended to cover various arrangements included within the spirit and scope of the broadest interpretation and equivalent arrangements.

What is claimed is:

1. A data storage device for integrating data of several medical measuring instruments, comprising:
a housing;
a control unit, installed in said housing, for controlling the action of each unit;
a transmission unit, electrically coupled to said control unit for receiving data from an external measuring instrument, and sending the external data to said control unit;
a computation unit, electrically coupled to said control unit for processing and comparing data;
a display unit, electrically coupled to said control unit for displaying data;
a reminding unit, electrically coupled to said control unit for issuing a warning;
a data storage unit, electrically coupled to said control unit for storing data received from the transmission unit and the data processed by the computation unit; and
a timing unit, electrically coupled to said control unit for providing the calculating time.

2. The data storage device for integrating data of several medical measuring instruments of claim 1, wherein said transmission unit is an infrared transmission interface.

3. The data storage device for integrating data of several medical measuring instruments of claim 1, wherein said control unit is a microprocessor.

4. The data storage device for integrating data of several medical measuring instruments of claim 1, wherein said display unit is a LCD monitor.

5. The data storage device for integrating data of several medical measuring instruments of claim 1, wherein said reminding unit is an alarm.

6. The data storage device for integrating data of several medical measuring instruments of claim 1, wherein said timing unit is a timer.
